(19) United States
(12) Patent Application Publication
(10) Pub. No.: US 2026/0166214 A1
(43) Pub. Date: Jun. 18, 2026

(54) NEGATIVE PRESSURE THERAPY DEVICE AND METHOD FOR CONTROLLING SAME

(71) Applicant: CG BIO CO., LTD., Seoul (KR)

(72) Inventors: Dong Ki Lee, Suwon-si Gyeonggi-do (KR); Gi Hun Kang, Yeongdong-gun Chungcheongbuk-do (KR); A Reum Park, Seongnam-si, Gyeonggi-do (KR)

(21) Appl. No.: 19/125,786

(22) PCT Filed: Nov. 29, 2023
(86) PCT No.: PCT/KR2023/019405

§ 371 Date: Apr. 30, 2025

(30) Foreign Application Priority Data
Nov. 30, 2022 (KR) 10-2022-0164000

(51) Int. Cl. A61M 1/00; A61M 39/00; A61M 39/22

(52) U.S. Cl. CPC A61M 1/966; A61M 1/60; A61M 1/98; A61M 39/223; A61M 2039/0009; A61M 2205/3344; A61M 2205/3358

(57) ABSTRACT

The present invention relates to a negative pressure therapy device and a method for controlling the same in which a pressure at a wound site is checked using a pressure sensor disposed on a valve side during an operation of applying a negative pressure to the wound site and the pressure at the wound site is checked using a pressure sensor disposed on a negative pressure generator side during supply of atmospheric air to the wound site through a valve, thereby reducing a pressure error rate caused by a flow rate.

### TECHNICAL FIELD

The present invention relates to a negative pressure therapy device and a method for controlling the same.

### BACKGROUND ART

A therapy method using a negative pressure is performed to promote healing of a wound site. In the therapy method using a negative pressure, first, a curer places a foam dressing on a wound site.

In a state in which the foam dressing is placed, the curer attaches a film dressing and a suction head to the skin adjacent to the wound site to seal the wound site. In a state in which the wound site is sealed in this way, the curer makes one end of a drain tubing part communicate with a sealed space, formed between the film dressing and the wound site, through the suction head.

The curer operates a pump communicating with the other end of the drain tubing part to supply a negative pressure into the sealed space. Accordingly, exudate discharged from the wound is absorbed into the foam dressing, and the exudate absorbed into the foam dressing is discharged to the outside of the sealed space through the drain tubing part.

By removing the exudate from the wound site, there is an effect of promoting the formation of granulation tissue, and healing of the wound is promoted.

### DISCLOSURE

### Technical Problem

The present invention is directed to providing a negative pressure therapy device and a method for controlling the same in which a pressure at a wound site is checked using a pressure sensor connected to a valve side during an operation of applying a negative pressure to the wound site and the pressure at the wound site is checked using a pressure sensor on a negative pressure generator side during supply of atmospheric air to the wound site through an open valve, thereby reducing a pressure error rate caused by a flow rate.

The present invention is also directed to providing a negative pressure therapy device and a method for controlling the same in which a pressure at a wound site can more accurately reach a target pressure, and after the pressure reaches the target pressure, from a comparison of values of two pressure sensors, the pressure can be corrected back to the target pressure when the pressure increases/decreases.

The present invention is also directed to providing a negative pressure therapy device and a method for controlling the same in which blockage of a tubing part due to exudate or the like can be prevented.

The present invention is also directed to providing a negative pressure therapy device and a method for controlling the same in which, by measuring an atmospheric pressure and correcting a measured pressure during an operation, an influence of an operation space (inside a helicopter, on a mountain, etc.) where the negative pressure therapy device is located and the surrounding environment thereof can be minimized.

### Technical Solution

According to one aspect of the present invention for addressing the above objectives, there is provided a negative pressure therapy device including a suction head provided to cover a wound site, first and second tubing parts each connected to the suction head to be fluidly connected to the wound site, a negative pressure generator provided to provide a negative pressure to the wound site through the first tubing part, a valve connected to the second tubing part and provided to selectively open the second tubing part toward an atmospheric air side so that atmospheric air is delivered to the wound site through the second tubing part, a first pressure sensor disposed to measure a pressure in the first tubing part, a second pressure sensor disposed to measure a pressure in the second tubing part, and a controller provided to perform a negative pressure provision mode providing the negative pressure to the wound site by operating the negative pressure generator and a negative pressure release mode lowering the negative pressure by supplying the atmospheric air to the wound site and provided to control the negative pressure generator based on a value measured by the second pressure sensor and a preset target pressure of the wound site in the negative pressure provision mode.

Also, the controller may be provided to control the valve based on a value measured by the first pressure sensor and the preset target pressure of the wound site in the negative pressure release mode.

Also, the valve may be connected to each of the atmospheric air side, the second tubing part, and the second pressure sensor. Specifically, the valve may have three connection ports, and each connection port may be connected to one of the atmospheric air side, the second tubing part, and the second pressure sensor side. The valve may be a 3-way valve or may be a solenoid valve that operates electromagnetically.

Also, the valve may be provided to fluidly connect the atmospheric air side and the second tubing part and block connection between the second pressure sensor and the second tubing part in the negative pressure release mode. Also, the valve may be provided to block connection between the atmospheric air side and the second tubing part and fluidly connect the second pressure sensor and the second tubing part in the negative pressure provision mode. Specifically, the valve may operate in two operation modes and may fluidly connect two of the three connection ports in each operation mode.

For example, the valve may be provided to fluidly connect the atmospheric air side and the second tubing part and block the connection between the second pressure sensor and the second tubing part in the negative pressure release mode of the controller. At this time, the atmospheric air may pass through the valve and be introduced into the second tubing part, such outside air may be delivered to the wound site through the second tubing part, and accordingly, the negative pressure at the wound site may decrease. Also, as the connection between the second pressure sensor and the second tubing part is blocked, the second pressure sensor may become unable to measure the pressure in the second tubing part. On the other hand, the valve may block the connection between the atmospheric air side and the second tubing part and fluidly connect the second pressure sensor and the second tubing part in the negative pressure provision mode of the controller. At this time, the atmospheric air may not be able to be introduced into the second tubing part, and the second pressure sensor may become able to measure the pressure in the second tubing part.

Also, the controller may be provided to stop an operation of the negative pressure generator and compare the value measured by the first pressure sensor and the preset target pressure of the wound site when the atmospheric air is delivered to the wound site through the second tubing part in the negative pressure release mode. That is, the controller may control an operation of the valve based on the value measured by the first pressure sensor in the negative pressure release mode.

Also, the controller may control the valve to block the connection between the atmospheric air side and the second tubing part during the operation of the negative pressure generator in the negative pressure provision mode.

Also, the controller may be provided to perform a cycle operation mode varying the negative pressure applied to the wound site between a highest target pressure and a lowest target pressure.

At this time, the controller may be provided to stop the negative pressure generator after operating the negative pressure generator and reaching the highest target pressure and adjust the pressure to the lowest target pressure as the atmospheric air is supplied to the wound site through the second tubing part in the cycle operation mode.

Also, the controller may be provided to measure a pressure at the wound site through the second pressure sensor in a process of reaching the highest target pressure and measure the pressure at the wound site through the first pressure sensor in a process of reaching the lowest target pressure.

Also, the negative pressure therapy device may further include a third pressure sensor for measuring an atmospheric pressure.

Also, the controller may be provided to compare a difference between values measured by the second pressure sensor and the third pressure sensor with the target pressure in the negative pressure provision mode, and the controller may be provided to compare a difference between values measured by the first pressure sensor and the third pressure sensor with the target pressure in the negative pressure release mode.

Also, the negative pressure therapy device may further include a canister to which exudate is delivered from the wound site through the first tubing part. The canister may perform a function of storing the exudate suctioned from the wound site through the first tubing part, the first tubing part may connect the suction head and the negative pressure generator, and the canister may be disposed between the suction head and the negative pressure generator. The first pressure sensor may be provided in a first sensing line branched from the first tubing part in a section between the canister and the negative pressure generator. The first pressure sensor provided in the first sensing line may be provided to measure the pressure in the first tubing part.

Also, the second tubing part may connect the suction head and the valve, and the second tubing part may be provided to connect the suction head and the valve while disposed to pass through the canister. At this time, the atmospheric air side, a second sensing line in which the second pressure sensor is provided, and the second tubing part may each be connected to one of the connection ports of the valve.

In another embodiment, the second tubing part may be connected to the valve through the canister, and the second pressure sensor may be provided to measure the pressure in the second tubing part in a section between the canister and the valve. That is, the second pressure sensor may be provided in the second sensing line branched from the second tubing part in the section between the canister and the valve. At this time, the valve may have two connection ports, and each connection port may be connected to one of the second tubing part and the atmospheric air side. Specifically, when the valve is open, the atmospheric air side and the second tubing part may be connected, and the outside air may be introduced into the second tubing part. Also, when the valve is closed, the connection between the atmospheric air side and the second tubing part may be blocked.

Also, the valve may be provided to block the connection between the atmospheric air side and the second tubing part in the negative pressure provision mode and connect the atmospheric air side and the second tubing part in the negative pressure release mode.

Also, the controller may be provided to stop the operation of the negative pressure generator and compare the value measured by the first pressure sensor and the preset target pressure of the wound site when the atmospheric air is delivered to the wound site through the second tubing part in the negative pressure release mode.

Also, the controller may control the valve to block the connection between the atmospheric air side and the second tubing part during the operation of the negative pressure generator in the negative pressure provision mode.

Also, according to another aspect of the present invention, there is provided a method for controlling the negative pressure therapy device, the method including operating the negative pressure generator while comparing the value measured through the second pressure sensor and the target pressure in the negative pressure provision mode and comparing the value measured through the first pressure sensor and the target pressure in the negative pressure release mode.

Also, the connection between the atmospheric air side and the second tubing part may be blocked and the negative pressure generator may be operated in the negative pressure provision mode, and the negative pressure generator may be stopped and the atmospheric air side and the second tubing part may be fluidly connected in the negative pressure release mode.

Also, according to still another aspect of the present invention, there is provided a negative pressure therapy device including a suction head disposed to cover a wound site, first and second tubing parts each connected to the suction head to be fluidly connected to the wound site, a negative pressure generator provided to provide a negative pressure to the wound site through the first tubing part, a valve connected to the second tubing part and provided to selectively open the second tubing part toward an atmospheric air side so that atmospheric air is delivered to the wound site through the second tubing part, a first pressure sensor disposed to measure a pressure in the first tubing part, a second pressure sensor disposed to measure a pressure in the second tubing part, and a controller provided to control the negative pressure generator and the valve based on a value measured by the first pressure sensor or the second pressure sensor and a preset target pressure of the wound site and provided to compare the value measured through the second pressure sensor and the target pressure during an operation of the negative pressure generator.

Also, the controller may be provided to compare the value measured through the first pressure sensor and the target pressure when the valve is open.

Also, the controller may be provided to stop the operation of the negative pressure generator when the valve is open.

Also, the controller may be provided to keep the valve closed during the operation of the negative pressure generator.

Also, the controller may be provided to perform a cycle operation mode varying the pressure applied to the wound site between a highest target pressure and a lowest target pressure.

Also, the controller may be provided to stop the negative pressure generator after operating the negative pressure generator and reaching the highest target pressure while the valve is closed and adjust the pressure to the lowest target pressure as the atmospheric air is supplied to the wound site through the second tubing part by opening the valve.

Also, the controller may be provided to measure the pressure at the wound site through the second pressure sensor during the operation of the negative pressure generator.

Also, the controller may be provided to measure the pressure at the wound site through the first pressure sensor when the valve is open.

Also, the negative pressure therapy device may further include a third pressure sensor for measuring an atmospheric pressure.

Also, the controller may be provided to compare a difference between values measured by the second pressure sensor and the third pressure sensor with the target pressure during the operation of the negative pressure generator.

Also, the controller may be provided to compare a difference between values measured by the first pressure sensor and the third pressure sensor with the target pressure when the valve is open.

Also, the negative pressure therapy device may further include a canister to which exudate is delivered from the wound site through the first tubing part.

Also, the controller may be provided to stop the operation of the negative pressure generator after the target pressure is reached, open the valve for a predetermined amount of time, close the valve after passage of the predetermined amount of time, and then re-operate the negative pressure generator to reach the target pressure.

Also, according to yet another aspect of the present invention, there is provided a method for controlling the negative pressure therapy device having the above-described structure, the method including operating the negative pressure generator while comparing the value measured through the second pressure sensor and the target pressure.

Also, the method may include, after the target pressure is reached, stopping the negative pressure generator and measuring the pressure at the wound site through each of the first pressure sensor and the second pressure sensor.

In addition, the method may include stopping the negative pressure generator and opening the valve to supply the atmospheric air to the wound site, and when the valve is open, the value measured through the first pressure sensor and the target pressure may be compared.

### Advantageous Effects

A negative pressure therapy device and a method for controlling the same according to one embodiment of the present invention have the following effects.

By checking a pressure at a wound site using a pressure sensor connected to a valve side during an operation of applying a negative pressure to the wound site and checking the pressure at the wound site using a pressure sensor connected to a negative pressure generator side during supply of atmospheric air to the wound site through an open valve, a pressure error rate caused by a flow rate can be reduced.

Also, a pressure at a wound site can more accurately reach a target pressure, and after the pressure reaches the target pressure, from a comparison of values of two pressure sensors, the pressure can be adjusted back to the target pressure when the pressure increases or decreases relative to the target pressure.

Also, blockage of a tubing part due to exudate or the like can be prevented.

In addition, by measuring an atmospheric pressure and correcting a measured pressure during an operation, an influence of an operation space (inside a helicopter, on a mountain, etc.) where the negative pressure therapy device is located and the surrounding environment thereof can be minimized.

### DESCRIPTION OF DRAWINGS

FIG. 1 is a schematic view for describing a suction head constituting a negative pressure therapy device according to one embodiment of the present invention.

FIG. 2 is a time-pressure (negative pressure) graph for describing a method for controlling the negative pressure therapy device according to one embodiment of the present invention.

FIG. 3 is a schematic view showing a negative pressure therapy device according to a first embodiment of the present invention.

FIGS. 4 to 6 are schematic views for describing one operational state of the negative pressure therapy device according to the first embodiment of the present invention.

FIG. 7 is a schematic view showing a negative pressure therapy device according to a second embodiment of the present invention.

FIGS. 8 and 9 are schematic views for describing one operational state of the negative pressure therapy device according to the second embodiment of the present invention.

### MODES OF THE INVENTION

Hereinafter, a negative pressure therapy device and a method for controlling the same according to one embodiment of the present invention will be described in detail with reference to the accompanying drawings.

The present invention may be implemented in various different forms and is not limited to embodiments described with reference to the drawings. Also, the same or corresponding components will be denoted by the same or similar reference numerals throughout the drawings, and redundant description thereof will be omitted, and the size and shape of each component illustrated in the drawings may be exaggerated or reduced for convenience of description.

Throughout the specification, when a certain portion is described as being “connected to (linked to, in contact with, coupled to)” another portion, this includes not only the case in which the two portions are “directly connected” but also the case in which the two portions are “indirectly connected” with another component disposed therebetween.

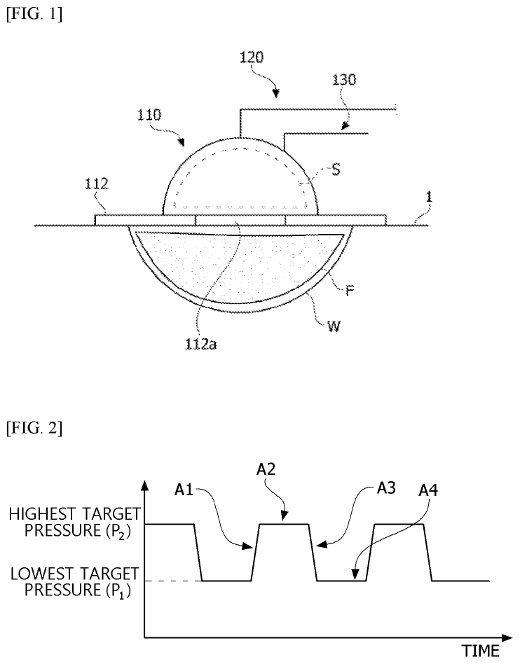

FIG. 1 is a schematic view for describing a suction head 110 constituting a negative pressure therapy device according to one embodiment of the present invention, and FIG. 2 is a time-pressure (negative pressure) graph for describing a method for controlling the negative pressure therapy device according to one embodiment of the present invention.

A negative pressure therapy device 100 according to one embodiment of the present invention includes the suction head 110, a first tubing part 120, and a second tubing part 130.

Referring to FIG. 1, in a state in which a foam dressing F is placed on a wound site W, a user may attach a film dressing 112 and the suction head 110 to skin 1 adjacent to the wound site W to seal the wound site W.

The film dressing 112 is used for the purpose of sealing the wound site W, and the film dressing 112 may be integrally formed with the suction head 110, or the suction head 110 may be provided to be attached to the film dressing 112 after the film dressing 112 is attached to the skin first. The film dressing 112 may have a multi-layer structure in which a plurality of functional films are stacked and may have a sticky layer or an adhesive layer provided on a surface facing the wound site. Also, the film dressing 112 has a through-hole 112a.

The suction head 110 has a space S fluidly connected to the through-hole 112a of the film dressing 112. Also, the first tubing part 120 and the second tubing part 130 are each fluidly connected to the space S in the suction head 110. The first tubing part 120 and the second tubing part 130 may be connected to different positions on the suction head 110.

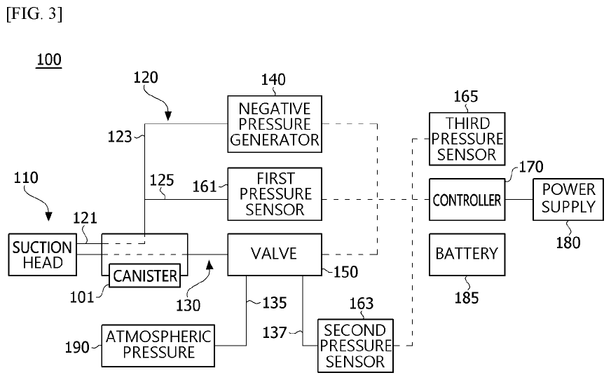

Also, FIG. 3 is a schematic view showing the negative pressure therapy device 100 according to a first embodiment of the present invention.

The negative pressure therapy device 100 according to the first embodiment of the present invention includes the suction head 110, the first tubing part 120, the second tubing part 130, a negative pressure generator 140, a valve 150, a first pressure sensor 161, a second pressure sensor 163, and a controller 170.

The negative pressure generator 140 performs a function of applying vacuum to the wound site W through the first tubing part 120 to reach a negative pressure state and is provided to adjust the negative pressure applied to the wound site W. The negative pressure generator 140 may include a pump for applying the negative pressure to the wound site.

In such a structure, the negative pressure generator 140 connected to the first tubing part 120 may be operated to supply the negative pressure to the wound site W sealed by the film dressing 112. Accordingly, exudate discharged from the wound site W is absorbed into the foam dressing F, and the exudate absorbed into the foam dressing F moves to the space S in the suction head 110 through the through-hole 112a of the film dressing 112, may be discharged to the outside of the wound site W through the first tubing part 120, moves toward the negative pressure generator 140 along the first tubing part 120, and is stored in a canister 101 which will be described below.

In this way, the negative pressure therapy device 100 according to one embodiment of the present invention is a device for sucking in the exudate or the like of the wound site W using a vacuum pressure of the pump, which is the negative pressure generator 140, and may be used for the purpose of sucking in blood, body fluids, or foreign matter in addition to the exudate.

Referring to FIGS. 1 and 3, the negative pressure therapy device 100 according to the first embodiment includes the suction head 110 provided to cover the wound site W and the first and second tubing parts 120 and 130 each connected to the suction head to be fluidly connected to the wound site W.

In addition, the negative pressure therapy device 100 includes the negative pressure generator 140 provided to provide a negative pressure to the wound site W through the first tubing part 120.

The first tubing part 120 performs a function of transmitting the vacuum pressure formed in the negative pressure generator 140 to the wound site through the suction head 110 and may have a structure in which a plurality of tubes are fluidly connected along a path in which the vacuum pressure is transmitted from the negative pressure generator 140 to the space S in the suction head 110.

In addition, the negative pressure therapy device 100 may include the valve 150 connected to the second tubing part 130 and provided to selectively open the second tubing part 130 toward an atmospheric air 190 side to deliver atmospheric air 190 to the wound site through the second tubing part 130.

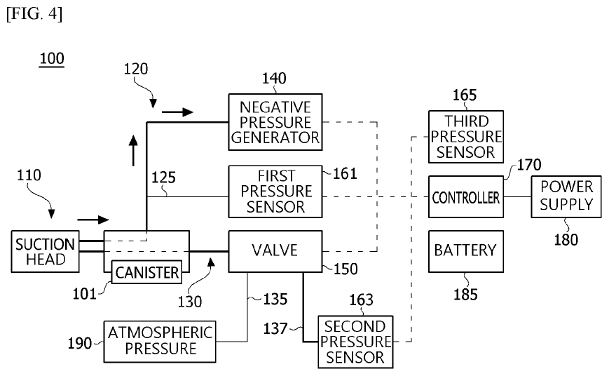

FIGS. 4 to 6 are schematic views for describing one operational state of the negative pressure therapy device according to one embodiment of the present invention.

The valve 150 may be an electromagnetic valve, and for example, the valve 150 may be a solenoid valve. The valve 150 may be a 3-way valve, may have three connection ports, and may be operated in two operation modes selectively connecting two of the three connection ports.

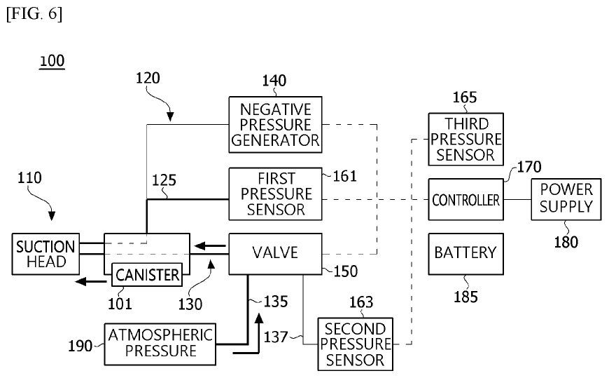

When the valve 150 is in a first operation mode (see FIG. 6), the second tubing part 130 may be open to the atmospheric air 190 side (the second tubing part and the atmospheric air side may be connected), and the atmospheric air may be delivered to the wound site W through the second tubing part 130 and the space S of the suction head 110. At this time, as the atmospheric air is supplied to the wound site W, the negative pressure acting on the wound site W may be adjusted. That is, the negative pressure in the wound site W formed by the negative pressure generator 140 may be adjusted (the size of the negative pressure may be decreased) by the atmospheric air introduced from the outside. On the other hand, when the valve 150 is in a second operation mode (see FIG. 4), the second tubing part 130 is not open toward the atmospheric air 190 side, and the second tubing part 130 remains fluidly connected to the first tubing part 120 through the space S in the suction head 110.

In addition, the negative pressure therapy device 100 includes the first pressure sensor 161 disposed to measure a pressure in the first tubing part 120 and the second pressure sensor 163 disposed to measure a pressure in the second tubing part 130. The first pressure sensor 161 may be provided in a first sensing line 125 branched from the first tubing part 120, and the second pressure sensor 163 may be provided in a second sensing line 163 connected to a connection port of the valve 150. The first pressure sensor 161 may measure the pressure in the first tubing part 120 due to being provided in the first sensing line 125 fluidly connected to the first tubing part 120. In addition, the second pressure sensor 161 may be provided in the second sensing line 137 connected to the connection port of the valve 150.

In addition, the valve 150 may be provided to operate in the first operation mode in a negative pressure release mode for the wound site W and operate in the second operation mode in a negative pressure provision mode for the wound site W.

The valve 150 may be provided to fluidly connect the atmospheric air side 190 and the second tubing part 130 and block connection between the second pressure sensor 163 and the second tubing part 130 in the first operation mode. At this time, the atmospheric air may pass through the valve 150 and be introduced into the second tubing part 130, such outside air may be delivered to the wound site W through the second tubing part 130, and accordingly, the negative pressure at the wound site W may decrease. Also, as the connection between the second sensing line 137, in which the second pressure sensor 163 is provided, and the second tubing part 130 is blocked, the second pressure sensor 163 becomes unable to measure the pressure in the second tubing part 130.

On the other hand, the valve 150 may block the connection between the atmospheric air side 190 and the second tubing part 130 and fluidly connect the second pressure sensor 163 and the second tubing part 130 in the second operation mode. At this time, the atmospheric air becomes unable to be introduced into the second tubing part 130, and the second pressure sensor 163 becomes able to measure the pressure in the second tubing part 130 as the second sensing line 137 and the second tubing part 130 are connected.

In addition, the negative pressure therapy device 100 includes the controller 170 provided to control the negative pressure generator 140 and the valve 150 based on a value measured by the first pressure sensor 161 or the second pressure sensor 163 and a preset target pressure of the wound site W.

In the present document, the term “target pressure” refers to a target negative pressure acting on the wound site W, and the controller 170 controls the negative pressure generator 140 and the valve 150 for the pressure in the wound site W to reach the target negative pressure. In addition, the term “operating pressure” refers to a negative pressure acting on the wound site W at the time of measurement, and the controller 170 controls the negative pressure generator 140 and the valve 150 for the operating pressure measured through the first pressure sensor 161 or the second pressure sensor 163 to reach the target pressure.

The controller 170 may be provided to perform the negative pressure provision mode operating the negative pressure generator 140 to provide a negative pressure to the wound site W and the negative pressure release mode supplying the atmospheric air to the wound site W to lower the negative pressure.

Referring to FIG. 4, the controller 170 may be provided to control the negative pressure generator based on a value measured by the second pressure sensor 163 and a preset target pressure of the wound site W in the negative pressure provision mode. In the present document, controlling the negative pressure generator 140 may mean operating the negative pressure generator 140 or stopping the operation thereof. In addition, controlling the valve 150 may mean changing the operation mode of the valve 150.

The controller 170 is provided to compare the value (operating pressure) measured through the second pressure sensor 163 and the target pressure during the operation of the negative pressure generator 140. In addition, the controller 170 may control the valve 150 and operate the valve 150 in the second operation mode during the operation of the negative pressure generator 140. At this time, the connection between the atmospheric air side 190 and the second tubing part 130 is blocked, and the second pressure sensor 163 and the second tubing part 130 are fluidly connected.

When the negative pressure generator 140 is operated, a flow rate is formed toward the negative pressure generator 140 in the first tubing part 120, and the operating pressure in the wound site W increases (a negative pressure value increases) until reaching the target pressure.

On the other hand, referring to FIG. 6, the controller 170 may operate the valve 150 in the first operation mode in the negative pressure release mode. At this time, the atmospheric air side 190 and the second tubing part 130 are fluidly connected, and the connection between the second pressure sensor 163 and the second tubing part 130 is blocked. At this time, the controller 170 may be provided to compare the value (operating pressure) measured through the first pressure sensor 161 and the target pressure. In addition, the controller 170 may be provided to stop the operation of the negative pressure generator 140 when the valve 150 operates in the first operation mode (negative pressure release mode).

When the negative pressure generator 140 is stopped and the valve 150 is operated in the first operation mode, introduction of atmospheric air toward the suction head 110 occurs in the second tubing part 130, and accordingly, a flow rate is formed toward the suction head 110 in the second tubing part 130, and the operating pressure in the wound site W decreases (the negative pressure value decreases) due to the introduced atmospheric air.

In the present document, introducing atmospheric air into the suction head 110 means introducing air in the atmosphere of the space where the negative pressure therapy device 100 is located into the space S in the suction head 110 through the second tubing part 130.

At this time, in order to reduce a pressure error rate caused by the flow rate, the pressure at the wound site W is checked using the second pressure sensor 163 connected to the valve 150 during an operation of applying a negative pressure to the wound site W, and the pressure at the wound site W is checked using the first pressure sensor 161 disposed at the negative pressure generator 140 side during supply of atmospheric air to the wound site W through the valve 150.

As described above, the first pressure sensor 161 may be provided to measure the pressure in the first tubing part 120 between the suction head 110 and the negative pressure generator 140, and the second pressure sensor 163 may be connected to the valve 150 and provided to measure the pressure in the second tubing part 130.

The controller 170 may be provided to perform a continuous operation mode maintaining the wound site W at a certain target pressure and a cycle operation mode varying the negative pressure applied to the wound site W between a highest target pressure P2 and a lowest target pressure P1 (see FIG. 2). FIG. 2 is a graph showing the cycle operation mode. For example, the highest target pressure P2 may be determined within a range of −120 mmHg to −200 mmHg, and when the highest target pressure P2 is −120 mmHg, the lowest target pressure P1 may range from −60 mmHg to −105 mmHg.

In addition, in the present document, the target pressure in the wound site W in the negative pressure provision mode and the negative pressure release mode and the highest target pressure P2 and the lowest target pressure P1 in the cycle operation mode may be arbitrarily set by a user or may be preset in the controller 170.

FIGS. 4 to 6 are schematic views for describing one operational state of the negative pressure therapy device according to the first embodiment of the present invention.

Referring to FIGS. 2 and 4, the controller 170 is provided to perform the negative pressure provision mode in section A1 (the section in which the highest target pressure is reached). At this time, the valve 150 is controlled to operate in the second operation mode, and the controller 170 may cause the wound site W to reach the highest target pressure P2 by operating the negative pressure generator 140. At this time, the controller 170 may control the negative pressure generator 140 while comparing the negative pressure at the wound site W measured through the second pressure sensor 163 and the highest target pressure.

In addition, after the highest target pressure P2 is reached, the controller 170 may maintain the highest target pressure P2 for a predetermined amount of time in section A2.

Then, the controller 170 may be provided to stop the negative pressure generator 140 and adjust the pressure to the lowest target pressure P1 as the atmospheric air is supplied to the wound site W through the second tubing part 130 by controlling the valve 150 to operate in the first operation mode in section A3 (the section in which the lowest target pressure is reached).

As described above, the pressure at the wound site W may be provided to be measured through the second pressure sensor 163 during the operation of the negative pressure generator 140 in section A1 (the section in which the highest target pressure is reached). That is, the controller 170 may perceive the operating pressure at the wound site W through the value of the second pressure sensor 163 and may control the negative pressure generator 140 so that the operating pressure reaches the target pressure.

Referring to FIGS. 2 and 6, the pressure at the wound site W may be provided to be measured through the first pressure sensor 161 during the operation of the valve 150 in the first operation mode in section A3 (the section in which the lowest target pressure is reached). That is, the controller 170 may perceive the operating pressure at the wound site W through the value of the first pressure sensor 161 and may control the valve 150 so that the operating pressure reaches the target pressure.

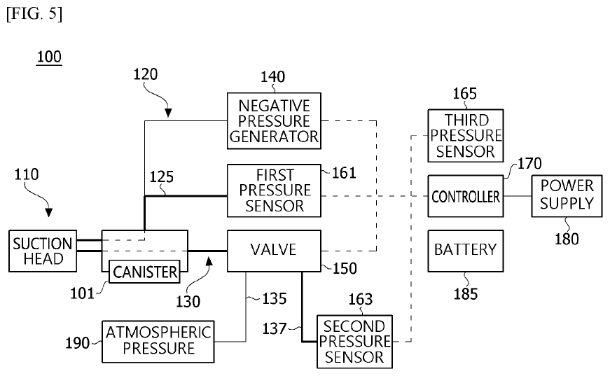

Referring to FIGS. 2 and 5, when the highest target pressure P2 is reached in the cycle operation mode, the controller 170 may maintain the highest target pressure P2 for a predetermined amount of time, and in section A2 (the section in which the highest target pressure is maintained), the controller 170 may be provided to stop the negative pressure generator 140 (maintain the valve in the second operation mode) and measure the pressure at the wound site W through each of the first pressure sensor 161 and the second pressure sensor 163. At this time, when the values measured through the first pressure sensor 161 and the second pressure sensor 163 are different, the controller 170 may be provided to perceive the operating pressure in the wound site W by prioritizing the value measured by the first pressure sensor 161. That is, when the values measured through the first pressure sensor 161 and the second pressure sensor 163 are different, the controller 170 perceives the operating pressure in the wound site W using the value measured by the first pressure sensor 161.

In addition, in section A2 (or section A4) of FIG. 2, when the operating pressure changes due to factors such as a leak occurring at the suction head 110 side and is lower than the target pressure, the controller 170 may operate the negative pressure generator 140 again so that the operating pressure reaches the target pressure again, and when the operating pressure changes and is higher than the target pressure, the controller 170 may operate the valve 150 in the first operation mode and introduce the atmospheric air into the wound site W so that the operating pressure reaches the target pressure again.

In addition, in section A, after the lowest target pressure P1 is reached, the controller 170 may maintain the lowest target pressure P1 for a predetermined amount of time.

Referring to FIGS. 2 and 5, when the lowest target pressure P1 is reached in the cycle operation mode, the controller 170 may maintain the lowest target pressure P1 for a predetermined amount of time, and in section A4 (the section in which the lowest target pressure is maintained), the controller 170 may be provided to maintain the valve 150 in the second operation mode, stop the negative pressure generator 140, and measure the pressure at the wound site W through each of the first pressure sensor 161 and the second pressure sensor 163. At this time, when the values measured through the first pressure sensor 161 and the second pressure sensor 163 are different, the controller 170 may be provided to perceive the operating pressure in the wound site W by prioritizing the value measured by the second pressure sensor 163.

In addition, in section A4, when the operating pressure changes due to factors such as a leak occurring at the suction head 110 side and is lower than the target pressure, the controller 170 may operate the negative pressure generator 140 so that the operating pressure reaches the target pressure again, and when the operating pressure changes and is higher than the target pressure, the controller 170 may operate the valve 150 in the first operation mode and introduce the atmospheric air into the wound site W so that the operating pressure reaches the target pressure again.

In addition, the negative pressure therapy device 100 may further include a third pressure sensor 165 for measuring an atmospheric pressure. The third pressure sensor 165 is a sensor measuring the atmospheric air of the location where the negative pressure therapy device 100 is placed, and serves to measure an atmospheric pressure value and adjust a zero point. That is, atmospheric pressure that was 1 atm at the base of a mountain gradually decreases toward the top of the mountain, and accordingly, the current pressure may be allowed to accurately reach the target pressure flexibly according to the environment based on a pressure value measured through the third pressure sensor 165.

In such a structure, the controller 170 may be provided to compare a difference between values measured by the second pressure sensor and the third pressure sensor with the target pressure during the operation of the negative pressure generator 140 (the negative pressure provision mode). That is, in order to increase the operating pressure to the target pressure as in section A1, during the operation of the negative pressure generator 140, the controller 170 may control the negative pressure generator 140 by recognizing the difference between the values measured by the second pressure sensor 163 and the third pressure sensor 165 as the operating pressure and comparing the operating pressure with the target pressure.

In addition, the controller 170 may be provided to operate the valve 150 in the first operation mode and compare a difference between values measured by the first pressure sensor 161 and the third pressure sensor 165 with the target pressure in the negative pressure release mode. That is, in order to decrease the operating pressure to the target pressure as in section A3, when the valve 150 is open, the controller 170 may control the valve 150 by recognizing the difference between the values measured by the first pressure sensor 161 and the third pressure sensor 165 as the operating pressure and comparing the operating pressure with the target pressure.

Referring to FIG. 3, the negative pressure therapy device 100 may include the canister 101 to which exudate is delivered from the wound site W through the first tubing part 120. In such a structure, the canister 101 may be disposed between the suction head 110 and the negative pressure generator 140. In addition, the first tubing part 120 may include a first tube 121 connecting the suction head 110 and the canister 200 and a second tube 123 connecting the canister 101 and the negative pressure generator 140. At this time, the exudate from the wound site W is stored in the canister 101 through the first tube 121. In addition, during the operation of the negative pressure generator 140, a sucking pressure is applied to the inside of the canister 101 through the second tube 123, and subsequently, the sucking pressure is applied to the wound site W through the first tube 121. That is, the first tube 121 and the second tube 123 may be fluidly connected through a space in the canister where the exudate is stored. At this time, the first sensing line 125 is branched from the second tube 123.

In addition, one or more first filters may be provided at a point of the canister 101 that is connected to the second tube 123. The first filers serve to prevent the exudate stored in the canister 101 from being introduced into the negative pressure generator 140 or the second tube 123.

In addition, one or more second filters may be provided in the second tubing part 130. The second filters serve to filter contaminants in the atmospheric air 190 when the atmospheric air 190 is introduced into the wound site W.

Meanwhile, a phenomenon in which the exudate stagnates in the first tubing part 120 or the second tubing part 130 may occur. For example, in the negative pressure provision mode, exudate, blood, or pus is introduced into the canister 101 along the first tube 121 during rotation of the pump of the negative pressure generator 140, and the rotation of the pump is stopped when the operating pressure measured by the second pressure sensor 163 has reached the target pressure. At this time, a phenomenon in which the exudate stagnates in the first tube 121 may occur in some cases.

The controller 170 may be provided to perform a blockage resolving mode in the middle of the negative pressure provision mode or after the negative pressure provision mode is completed. For example, the controller 170 may be provided to perform the blockage resolving mode after the target pressure is reached through the negative pressure provision mode. In the blockage resolving mode, the controller 170 may stop the operation of the negative pressure generator 140 and may operate the valve 150 in the first operation mode for a predetermined amount of time. At this time, after passage of the predetermined amount of time, the controller 170 may be provided to switch the valve 150 to the second operation mode to perform the negative pressure provision mode and operate the negative pressure generator 140 again to reach the target pressure.

That is, in the negative pressure provision mode, by operating the valve 150 in the first operation mode every predetermined amount of time (for example, 5 minutes), the atmospheric air may be supplied to the inside of the suction head 110 to intentionally release the pressure from inside the first tube 121, and by rotating the pump again until the target pressure after a predetermined amount of time (for example, 1 minute), the exudate stagnating in the first tubing part 120 or the second tubing part 130 may be removed. That is, in this way, blockage in the first tubing part 120 or the second tubing part 130 may be resolved or prevented.

In addition, the negative pressure therapy device 100 may include a power supply 180 for receiving external power. In addition, the negative pressure therapy device 100 may include a battery 185. When the battery 185 is used, even without supply of external power, the negative pressure therapy device 100 may be used for a predetermined amount of time through power charged in the battery 185.

In addition, according to still another aspect of the present invention, a method for controlling the negative pressure therapy device 100 having the structure described above relates to the cycle operation mode. The method for controlling the negative pressure therapy device according to one embodiment of the present invention includes operating the negative pressure generator 140 while comparing the value (operating pressure) measured through the second pressure sensor 163 and the target pressure. At this time, the valve 150 may be operated in the second operation mode so that the atmospheric air is not introduced into the space in the suction head 110 (refer to section A1).

In addition, the method may include, after the target pressure is reached, stopping the negative pressure generator 140 and measuring the pressure at the wound site through each of the first pressure sensor 161 and the second pressure sensor 163 (refer to section A2).

In addition, the method may include stopping the negative pressure generator 140 and operating the valve 150 in the first operation mode to supply the atmospheric air to the wound site (refer to section A3). At this time, the value measured through the first pressure sensor 161 and the target pressure may be compared.

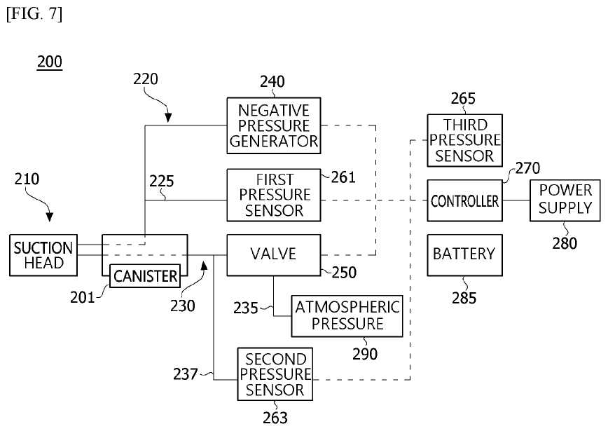

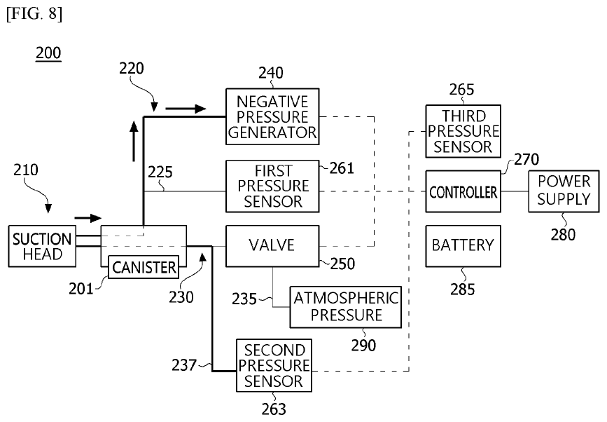

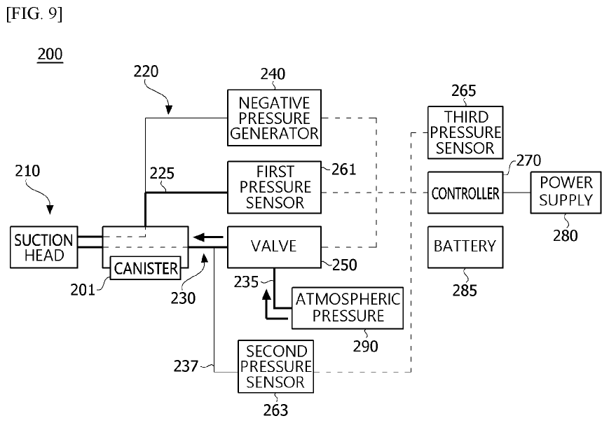

FIG. 7 is a schematic view showing a negative pressure therapy device 200 according to a second embodiment of the present invention, and FIGS. 8 and 9 are schematic views for describing one operational state of the negative pressure therapy device 200 according to the second embodiment of the present invention.

The negative pressure therapy device 200 according to the second embodiment is different from the negative pressure therapy device 100 according to the first embodiment in terms of the type of a valve 250 and a position at which a second pressure sensor 263 is connected.

The negative pressure therapy device 200 according to the second embodiment of the present invention includes a suction head 210, a first tubing part 220, a second tubing part 230, a negative pressure generator 240, the valve 250, a first pressure sensor 261, the second pressure sensor 263, and a controller 270.

Referring to FIG. 7, the negative pressure therapy device 200 includes the suction head 210 disposed to cover a wound site W and the first and second tubing parts 220 and 230 each connected to the suction head 210 to be fluidly connected to the wound site W. In addition, the negative pressure therapy device 200 includes the negative pressure generator 240 provided to provide a negative pressure to the wound site through the first tubing part 220.

In addition, the negative pressure therapy device 200 may include the valve 250 connected to the second tubing part 230 and provided to selectively open the second tubing part 230 toward an atmospheric air 290 side to deliver atmospheric air 290 to the wound site through the second tubing part 230.

The valve 250 may be an electromagnetic valve, and for example, the valve 250 may be a solenoid valve. In a state in which the valve 250 is open, the second tubing part 230 may be open toward the atmospheric air side 190, and the atmospheric air may be delivered to the wound site W through the second tubing part 230 and a space S of the suction head 210. At this time, as the atmospheric air is supplied to the wound site W, the negative pressure acting on the wound site W may be adjusted. That is, the negative pressure in the wound site W formed by the negative pressure generator 240 may be adjusted (the size of the negative pressure may be decreased) by the introduced atmospheric air. On the other hand, in a state in which the valve 250 is closed, the second tubing part 230 is not open toward the atmospheric air 290 side, and the second tubing part 230 remains fluidly connected to the first tubing part 220 through the space S (see FIG. 1) in the suction head 210.

In addition, the negative pressure therapy device 200 includes the first pressure sensor 261 disposed to measure a pressure in the first tubing part 220 and the second pressure sensor 263 disposed to measure a pressure in the second tubing part 230.

The negative pressure therapy device 200 may include a canister 201 to which exudate is delivered from the wound site W through the first tubing part 220. In such a structure, the canister 201 may be disposed between the suction head 210 and the negative pressure generator 240.

In addition, the first pressure sensor 261 may be provided to measure the pressure in the first tubing part 220 between the suction head 210 and the negative pressure generator 240, and for example, the first pressure sensor 261 may be provided in a first sensing line 225 branched from the first tubing part 220 in a section between the canister 201 and the negative pressure generator 240. In addition, the second pressure sensor 163 may be provided to measure the pressure in the second tubing part 230 between the suction head 210 and the valve 250. For example, the second pressure sensor 263 may be provided in a second sensing line 237 branched from the second tubing part 230 in a section between the canister 201 and the valve 250.

Meanwhile, the undescribed reference numeral 235 may be a connection line connected to the valve 250 or a connection port of the valve 250 for connecting the second tubing part 230 to the atmospheric air side 290. That is, when the valve 250 is open, the atmospheric air 290 may be introduced into the second tubing part 230 after passing through the valve 250 through the connection line or the connection port.

Referring to FIG. 8, the controller 270 is provided to compare the value (operating pressure) measured through the second pressure sensor 263 and the target pressure during the operation of the negative pressure generator 240 in the negative pressure provision mode. In addition, the controller 270 may be provided to keep the valve 250 closed during the operation of the negative pressure generator 240.

At this time, when the negative pressure generator 240 is operated, a flow rate is formed toward the negative pressure generator 240 in the first tubing part 220, and the operating pressure in the wound site W increases (a negative pressure value increases) until reaching the target pressure.

Referring to FIG. 9, the controller 270 may be provided to compare the value (operating pressure) measured through the first pressure sensor 261 and the target pressure when the valve 250 is open in the negative pressure release mode. In addition, the controller 270 may be provided to stop the operation of the negative pressure generator 240 when the valve 250 is open.

When the negative pressure generator 240 is stopped and the valve 250 is opened, introduction of atmospheric air toward the suction head 210 occurs in the second tubing part 230, and accordingly, a flow rate is formed toward the suction head 210, and the operating pressure in the wound site W decreases (the negative pressure value decreases) due to the introduced atmospheric air.

At this time, in order to reduce a pressure error rate caused by the flow rate, the pressure at the wound site is checked using the second pressure sensor 263 connected to the second tubing part 230 during an operation of applying a negative pressure to the wound site W, and the pressure at the wound site W is checked using the first pressure sensor 261 disposed at the negative pressure generator 240 side during supply of atmospheric air to the wound site W through the valve 250 that is open.

In addition, the negative pressure therapy device 200 may further include a third pressure sensor 265 for measuring an atmospheric pressure. The third pressure sensor 265 is a sensor measuring the atmospheric air of the location where the negative pressure therapy device 200 is placed, and serves to measure an atmospheric pressure value and adjust a zero point.

In addition, the negative pressure therapy device 200 may include a power supply 280 for receiving external power. In addition, the negative pressure therapy device 200 may include a battery 285. When the battery 285 is used, even without supply of external power, the negative pressure therapy device 200 may be used for a predetermined amount of time through power charged in the battery 285.

The exemplary embodiments of the present invention which have been described above are only disclosed for illustrative purposes. Those of ordinary skill in the art to which the present invention pertains may make various modifications, changes, and additions within the spirit and scope of the present invention, and such modifications, changes, and additions also belong to the scope of the claims below.

### Industrial Applicability

According to a negative pressure therapy device and a method for controlling the same according to one embodiment of the present invention, by checking a pressure at a wound site using a pressure sensor connected to a valve side during an operation of applying a negative pressure to the wound site and checking the pressure at the wound site using a pressure sensor connected to a negative pressure generator side during supply of atmospheric air to the wound site through an open valve, a pressure error rate caused by a flow rate can be reduced.

1. A negative pressure therapy device comprising:
a suction head provided to cover a wound site;
first and second tubing parts each connected to the suction head to be fluidly connected to the wound site;
a negative pressure generator provided to provide a negative pressure to the wound site through the first tubing part;
a valve connected to the second tubing part and provided to selectively open the second tubing part toward an atmospheric air side so that atmospheric air is delivered to the wound site through the second tubing part;
a first pressure sensor disposed to measure a pressure in the first tubing part;
a second pressure sensor disposed to measure a pressure in the second tubing part; and
a controller provided to perform a negative pressure provision mode providing the negative pressure to the wound site by operating the negative pressure generator and a negative pressure release mode lowering the negative pressure by supplying the atmospheric air to the wound site and provided to control the negative pressure generator based on a value measured by the second pressure sensor and a preset target pressure of the wound site in the negative pressure provision mode.

2. The negative pressure therapy device of claim 1, wherein the controller is provided to control the valve based on a value measured by the first pressure sensor and the preset target pressure of the wound site in the negative pressure release mode.

3. The negative pressure therapy device of claim 2, wherein:
the valve is connected to each of the atmospheric air side, the second tubing part, and the second pressure sensor;
the valve is provided to fluidly connect the atmospheric air side and the second tubing part and block connection between the second pressure sensor and the second tubing part in the negative pressure release mode; and
the valve is provided to block connection between the atmospheric air side and the second tubing part and fluidly connect the second pressure sensor and the second tubing part in the negative pressure provision mode.

4. The negative pressure therapy device of claim 3, wherein the controller is provided to stop an operation of the negative pressure generator and compare the value measured by the first pressure sensor and the preset target pressure of the wound site when the atmospheric air is delivered to the wound site through the second tubing part in the negative pressure release mode.

5. The negative pressure therapy device of claim 4, wherein the controller controls the valve to block the connection between the atmospheric air side and the second tubing part during the operation of the negative pressure generator in the negative pressure provision mode.

6. The negative pressure therapy device of claim 3, wherein:
the controller is provided to perform a cycle operation mode varying the negative pressure applied to the wound site between a highest target pressure and a lowest target pressure; and
the controller is provided to stop the negative pressure generator after operating the negative pressure generator and reaching the highest target pressure and adjust the pressure to the lowest target pressure as the atmospheric air is supplied to the wound site through the second tubing part in the cycle operation mode.

7. The negative pressure therapy device of claim 6, wherein the controller is provided to measure a pressure at the wound site through the second pressure sensor in a process of reaching the highest target pressure and measure the pressure at the wound site through the first pressure sensor in a process of reaching the lowest target pressure.

8. The negative pressure therapy device of claim 3, further comprising a third pressure sensor for measuring an atmospheric pressure.

9. The negative pressure therapy device of claim 8, wherein:
the controller is provided to compare a difference between values measured by the second pressure sensor and the third pressure sensor with the target pressure in the negative pressure provision mode; and
the controller is provided to compare a difference between values measured by the first pressure sensor and the third pressure sensor with the target pressure in the negative pressure release mode.

10. The negative pressure therapy device of claim 1, further comprising a canister to which exudate is delivered from the wound site through the first tubing part.

11. The negative pressure therapy device of claim 10, wherein:
the second tubing part is connected to the valve through the canister;
the second pressure sensor is provided to measure the pressure in the second tubing part in a section between the canister and the valve; and
the valve is provided to block the connection between the atmospheric air side and the second tubing part in the negative pressure provision mode and connect the atmospheric air side and the second tubing part in the negative pressure release mode.

12. The negative pressure therapy device of claim 11, wherein the controller is provided to stop the operation of the negative pressure generator and compare the value measured by the first pressure sensor and the preset target pressure of the wound site when the atmospheric air is delivered to the wound site through the second tubing part in the negative pressure release mode.

13. The negative pressure therapy device of claim 12, wherein the controller controls the valve to block the connection between the atmospheric air side and the second tubing part during the operation of the negative pressure generator in the negative pressure provision mode.

14. A method for controlling the negative pressure therapy device of claim 1, the method comprising:
operating the negative pressure generator while comparing the value measured through the second pressure sensor and the target pressure in the negative pressure provision mode; and
comparing the value measured through the first pressure sensor and the target pressure in the negative pressure release mode.

15. The method of claim 14, wherein the connection between the atmospheric air side and the second tubing part is blocked in the negative pressure provision mode, and the negative pressure generator is stopped in the negative pressure release mode.
